(19) United States
(12) Patent Application Publication
(10) Pub. No.: US 2008/0287434 A1
(43) Pub. Date: Nov. 20, 2008

(54) New amino-alkyl-amide derivatives as CCR3 receptor ligands

(75) Inventors: Agnes PAPPNE BEHR, Budapest (HU); Zoltan KAPUI, Budapest (HU); Peter ARANYI, Budapest (HU); Sandor BATORI, Budapest (HU); Veronika BARTANE BODOR, Budapest (HU); Lajos T. NAGY, (US); Mihalyne T. Santa, Mezotur (HU); Marton VARGA, Dunakeszi (HU); Gyorgy FERENCZY, Budapest (HU); Endre MIKUS, Budapest (HU); Katalin URBAN-SZABO, Budapest (HU); Judit VARGANE SZEREDI, Budapest (HU); Erzsebet WALCZ, Budapest (HU); Edit SUSAN, Dunakeszi (HU)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/050,969

(22) Filed: Mar. 19, 2008

Related U.S. Application Data
(63) Continuation of application No. PCT/HU2006/000078, filed on Sep. 19, 2006.

(30) Foreign Application Priority Data
Sep. 22, 2005 (HU) P0500878
Sep. 15, 2006 (HU) P0600726

(51) Int. Cl. A61K 31/5377; A61K 31/166; C07C 233/78; A61K 31/50; C07D 237/14; A61P 31/18; A61K 31/437; C07D 471/04; C07D 513/04

(52) U.S. Cl. 514/233.2; 564/185; 514/617; 514/247; 544/241; 546/120; 514/303; 544/127

(57) ABSTRACT

The invention relates to a compound of the general formula (I),

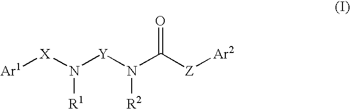

as defined herein which is useful for the treatment of a pathology in a patient wherein a CCR3 receptor plays a role in the development of the pathology, and pharmaceutical preparations containing such compound.

The invention is also directed to a process for preparing the compound of the general formula (I), and intermediate useful in the preparation.

### FIELD OF THE INVENTION

The present invention relates to the CCR3 receptor ligands of general formula (I), within them favourably antagonists and to the salts, solvates and isomers thereof, to the pharmaceutical compositions containing them, to the use of the compounds of the general formula (I) and their salts, solvates and isomers and to the preparation of the compounds of the general formula (I) and their salts, solvates and isomers.

### BACKGROUND OF THE INVENTION

Chemokines are small molecular weight (8-12 kDa) secreted polypeptides playing important regulatory role in the immune processes due to their leukocyte attracting (chemotactic) effect. They exert their effects through the chemokine receptors, which belong to the family of the G protein coupled receptors.

The CC chemokine receptors 3 (CCR3 receptors) are expressed by a number of inflammation cells, like the basofils, mast cells, T lymphocytes, epithelial cells, dendritic cells, but in the greatest amount they can be found on the surface of the eosinofils.

The CCR3 receptor ligands belong to the family of the C—C kemokines. They have a number of selective and non-selective ligands. The selective ligands are the eotaxin, eotaxin-2 and the lately discovered eotaxin-3. The non-selective ligands are the RANTES, the monocyte chemotactic proteins (MCP-2, MCP-3, MCP-4) and the macrophag inhibitor protein (MIP-1). The best characterized CCR3 ligand known from a long time is the eotaxin.

The eotaxin through the activation of the CCR3 receptors attracts selectively the eosinofils. Prior to an allergen provocation, the measured eotaxin level in the broncho-alveolar lavage fluidum of asthmatic patients was by 67 percent higher. On the effect of provocation a 2.4-fold increase of the epithelial and endothelial cells of the respiratory tract were found.

In the lung the eotaxin is produced in many cells. Following an allergen response, the most important eotaxin sources are the epithelial cells, but a great amount of eotaxin is produced by the fibroblasts of the lung, the smooth muscle cells and the endothelial cells of the respiratory tract, the alveolar macrophags and lymphocytes, and the eosinofils themselves.

Originally the data showed that the CCR3 receptors are to be found only in the eosinofil cells (Bertrand C P, Ponath P D., Expert Opin Investig Drugs. 2000 January; 9(1):43-52.), but on the basis of expression profiles it has been revealed that other inflammatory cells—although in smaller amount—also contain CCR3 receptors (Elsner J, Escher S E, Forssmann U., Allergy. 2004 December; 59(12):1243-58.). Thus, the CCR3 antagonists possess much wider effect, their activity is not limited to the eosinofils and consequently they can be considered much more valuable and effective targets in the treatment of asthmatic, allergic and inflammatory diseases.

Based on the above observations, CCR3 antagonists may possess important profilactic and therapeutic effects in the treatment of pathologies where in the development of the disease CCR3 receptors play a role. These diseases are characterized by the disorder of the leucocyte functions (activation, chemotaxis), there are numerous chronic inflammatory diseases among them, such as asthma, allergic rhinitis, atopic dermatitis, eczema, inflammatory bowel disease, ulcerative colitis, allergic conjunctivitis, arthritis, multiple sclerosis, Crohn's disease, HIV-infection and diseases in conjunction with AIDS.

The CCR3 antagonists published to date in the literature are carbamide-, thiocarbamide derivatives (WO 01/09088, WO 02/059081) and/or compounds containing saturated cyclic amino group (WO 00/35451, U.S. Pat. No. 6,605,623, WO 01/98270, WO 03/004487, WO 03/018556, WO 2004/028530, WO 00/53600, WO 00/35876, WO 01/64216, WO 02/50064, WO 02/102775, GB 2373186, WO 03/082291, WO 2004/004731, WO 2004/058702, WO 2004/085423). The present invention relates to a new structural type of compounds, to the open-chain amino-alkyl-amide derivatives, representatives of these compounds are effective CCR3 receptor antagonists.

From the aspect of therapeutic use it is essential that the molecules do not bind, or bind only in case of very high concentration to other the CCR receptor subtypes.

Our aim was to prepare compounds of high antagonistic activity, and at the same time selective to the CCR3 receptor, i.e. which inhibit the CCR3 receptor in much smaller concentration as compared to other CCR receptors. Further aim was that the new compounds have stability, bioavailability, therapeutic index and toxicity values which ensure its drugability. Additional aim was that the compounds, through their good enteric absorption can be applied orally.

### SUMMARY OF THE INVENTION

We have found that the compounds of the general formula (I),

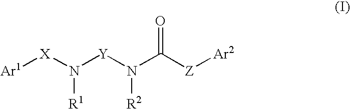

wherein
Ar¹ stands for phenyl group, optionally substituted with halogen atom;
X and Y independently mean straight C₁₋₄ alkylene group, optionally substituted with one or more identical or non-identical straight or branched C₁₋₄ alkyl group;
Z means valence bond or straight C₂₋₄ alkylene group or straight C₂₋₄ alkenylene group, optionally substituted with one or more identical or non-identical straight or branched C₁₋₄ alkyl group;
R¹ and R² independently mean hydrogen atom or straight or branched C₁₋₄ alkyl group;
Ar² stands for phenyl-, thienyl- or furyl group, each optionally substituted with one or more identical or non-identical substituents selected from the group consisting of straight or branched C₁₋₄ alkyl group, straight or branched C₁₋₄ alkoxy group, hydroxyl group, amino group, amino group—substituted with one or two identical or non-identical straight or branched C₁₋₄ alkyl group-, trifluoromethyl group, cyano group, C₁₋₂ alkylenedioxy group, and halogen atom;
   5- or 6-membered heterocyclic ring group containing one, two, or three nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, straight or branched C₁₋₄ alkoxy group, halogen atom, nitro group, cyano group, carboxyl group, phenyl group—optionally substituted with one or more straight or branched C₁₋₄ alkyl group, halogen atom, or benzyloxy group-, oxo group, —NR¹⁰R¹¹ group, —CONR¹⁰R¹¹ group, and —SO₂NR¹⁰R¹¹ group, wherein R¹⁰ and R¹¹ independently mean hydrogen atom, straight or branched C₁₋₄ alkyl group, C₃₋₆ cycloalkyl group, or benzyl group, or R¹⁰ and R¹¹ form together with the nitrogen atom a group of the general formula (a),

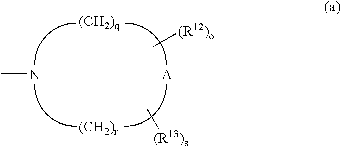

wherein
R¹² and R¹³ stand for hydrogen atom or straight or branched C₁₋₄ alkyl group,
A stands for methylene group, oxygen atom, sulphur atom, —NR¹⁴— group—wherein R¹⁴ stand for hydrogen atom, straight or branched C₁₋₄ alkyl group, C₃₋₆ cycloalkyl group or benzyl group-,
q represents zero, 1, 2, or 3,
r represents 1, or 2,
o represents zero, or 1,
s represents zero, or 1;
benzologue of the 5- or 6-membered heterocyclic ring group wherein the benzene ring may optionally be further substituted with one or more identical or non-identical substituents selected from the group consisting of halogen atom, straight or branched C₁₋₄ alkyl group, straight or branched C₁₋₄ alkoxy group, trifluoromethyl group, nitro group, cyano group, carboxyl group, C₁₋₂ alkylenedioxy group, hydroxyl group, sulfonyl group, —NR¹⁰R¹¹ group, —CONR¹⁰R¹¹ group, and —SO₂NR¹⁰R¹¹ group; or
5- or 6-membered heterocyclic ring group containing one, two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, cyano group, carboxyl group, hydroxyl group, —NR¹⁰R¹¹ group, —CONR¹⁰R¹¹ group, and —SO₂NR¹⁰R¹¹ group;
and their salts, solvates and isomers and the salts and solvates thereof fulfil the above criteria.

### DETAILED DESCRIPTION OF THE INVENTION

The detailed meanings of the above substituents are as follows:

By a C₁₋₄ alkyl group we mean a saturated straight- or branched-chain aliphatic group of 1-4 carbon atom, such as methyl-, ethyl-, propyl-, isopropyl-, butyl-, isobutyl-, secondary butyl-, tertiary butyl group.

By a C₁₋₄ alkylene group we mean a —(CH₂)ₙ— group where the value of n is 1, 2, 3 or 4, such as a methylene-, ethylene-, propylene-, butylene group.

By a C₂₋₄ alkenylene group we mean an alkenylene group containing 1 double bound, e.g. a —CH═CH— or —CH₂—CH═CH-group.

By a C₁₋₄ alkoxy group we mean an —O-alkyl group—where the meaning of alkyl is as defined above-, such as methoxy-, ethoxy-, propoxy-, isopropoxy-, butoxy-, isobutoxy-, secondary butoxy-, tertiary butoxy group.

By a C₁₋₂ alkylenedioxy group we mean an —O-alkylene-O— group—where the meaning of alkylene is as defined above-, such methylenedioxy-, ethylenedioxy group.

By halogen atom we mean chloro, fluoro, iodo or bromo atom.

By a 5- or 6-membered heterocyclic ring containing one, two or three nitrogen atoms we mean an unsaturated, saturated or partially saturated heterocyclic ring, for example pyrrole, imidazole, pyrazole, 1,2,3-triazole, 1,2,4-triazole, pyridine, pyrimidine, pyridazine, pyrazine 1,2,4-triazine, 1,3,5-triazine, 1,2,3-triazine, pyrrolidine, imidazolidine, [1,2,4]triazolidine, piperidine, piperazine, 2-imidazoline ring.

By a 5- or 6-membered heterocyclic ring containing one nitrogen atom and one oxygen or sulphur atom we mean an unsaturated, saturated or partially saturated heterocyclic ring, for example oxazole, isoxazole, thiazole, isothiazole, 1,2-oxazine, 1,3-oxazine, 1,4-oxazine, 1,2-thiazine, 1,3-thiazine, 1,4-thiazine, oxazolidine, thiazolidine, morpholine, thiomorpholine, 2-thiazoline, 2-oxazoline ring.

The heterocyclic ring containing two nitrogen atoms and one oxygen atom may be for example an oxadiazole ring.

By benzologue we mean derivatives condensed with benzene ring, for example indole, benzoxazole, benzthiazole, benzimidazole, quinoline, quinazoline, quinoxaline.

A derivative of a 5-6-membered heterocyclic ring—containing one, two or three nitrogen atoms, or one nitrogen atom and one oxygen atom, or one nitrogen atom and one sulphur atom—condensed with 6-membered heterocyclic rings—containing one or two nitrogen atom, may for example be a thiazolopyridine, triazolopyridine, thiazolopyrimidine, oxazolopyridine, 9H-purine, 3H-imidazopyridine.

The group of the general formula (a) preferably represents pyrrolidino, piperidino, piperazino, 4-methylpiperazino or morpholino group.

By salts of the compounds of general formula (I) we mean salts given with inorganic and organic acids and bases. Preferable are the salts formed with pharmaceutically acceptable acids e.g. hydrochloric acid, sulfuric acid, ethanesulfonic acid, tartaric acid, fumaric acid, citric acid, and bases e.g. sodium hydroxide, potassium hydroxide, ethanolamine. The salts formed during the purification and isolation process, favourably with tetrafluoroboric acid and perchloric acid, are also subjects of the invention.

By solvates we mean solvates formed with various solvents, e.g. with water or ethanol.

By isomers we mean structural and optical isomers. Structural isomers may be tautomeric forms in equilibrium or isolated desmotrops, which are also subjects of the invention. The compounds of general formula (I) may contain one or more asymmetric carbon atom, thus they may be optical isomers, enantiomers or diastereoisomers. These enantiomers and diastereoisomers and the mixtures thereof, including the racemates are also subjects of the invention.

A favourable group of the compounds of general formula (I) is formed by the compounds, where
Ar¹ stands for phenyl group, optionally substituted with one or more halogen atom;
X and Y independently mean straight C₁₋₄ alkylene group, optionally substituted with one or more identical or non-identical straight or branched C₁₋₄ alkyl group;
Z means straight C₂₋₄ alkylene group or C₂₋₄ alkenylene optionally substituted with one or more identical or non-identical straight or branched C₁₋₄ alkyl group;
R¹ and R² independently mean hydrogen atom or straight or branched C₁₋₄ alkyl group;
Ar² stands for phenyl group;
   5- or 6-membered heterocyclic ring containing one, two, or three nitrogen atoms, or one nitrogen atom and one oxygen atom, or one nitrogen atom and one sulphur atom, optionally substituted with one or more straight or branched C₁₋₄ alkyl group; benzologue of the 5- or 6-membered heterocyclic ring group where the benzene ring may optionally be further substituted with one or more identical or non-identical substituents selected from the group consisting of halogen atom, straight or branched C₁₋₄ alkyl group, amino group, and amino group—substituted with one or more identical or non-identical straight or branched C₁₋₄ alkyl group-; or
   5-membered heterocyclic ring group containing two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, —CONR¹⁰R¹¹ group, and —NR¹⁰R¹¹ group—wherein the meanings of R¹⁰ and R¹¹ are as defined above-; and their salts, solvates and isomers and the salts and solvates thereof.

Especially favourable are the following compounds:
3-(Benzothiazol-2-yl)1-N-{3-[(3,4-dichlorobenzyl)(methyl)amino]propyl}propanamide,
N-{3-[(3,4-dichlorobenzyl)(methyl)amino]propyl}-3-(6-methylbenzothiazol-2-yl)-propanamide,
N-{3-[(3,4-dichlorobenzyl)(methyl)amino]propyl}-3-(6-methylbenzoxazol-2-yl)propanamide,
3-(1H-Benzimidazol-2-yl)-N-{3-[(3,4-dichlorobenzyl)(methyl)amino]propyl}propanamide,
N-{3-[(3,4-dichlorobenzyl)(methyl)amino]propyl}-3-phenylpropanamide,
N-{3-[(3,4-dichlorobenzyl)(methyl)amino]propyl}-3-(7-methyl-[1,2,4]triazolo[1,5-a]pyridin-2-yl)propanamide,
N-{3-[(3,4-dichlorobenzyl)(methyl)amino]propyl}-3-(5-dimethylaminothiazolo[5,4-d]pyrimidin-2-yl)propanamide,
N-{3-[(3,4-dichlorobenzyl)(methyl)amino]propyl}-3-(5-dimethylaminothiazolo[5,4-b]pyridin-2-yl)propanamide,
N-{3-[(3,4-dichlorobenzyl)(methyl)amino]propyl}-3-(5-isopropylaminothiazolo[5,4-b]pyridin-2-yl)propanamide,
N-(3-{[1-(3,4-dichlorophenyl)ethyl]amino}propyl)-3-(5-methylamino[1,3]thiazolo[5,4-b]pyridin-2-yl)propanamide,
N-{3-[[1-(3,4-dichlorophenyl)ethyl])(methyl)amino]propyl}-3-(5-methylamino[1,3]thiazolo[5,4-b]pyridin-2-yl)propanamide,
N-{3-[[1-(3,4-dichlorophenyl)ethyl])(methyl)amino]propyl}-3-(5-piperidin-1-yl[1,3]thiazolo[5,4-b]pyridin-2-yl)propanamide,
N-{3-[[1-(3,4-dichlorophenyl)ethyl])(methyl)amino]propyl}-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-piperidin-1-yl[1,3]thiazolo[5,4-b]pyridin-2-yl)propanamide,
N-(3-{[1-(3,4-dichlorophenyl)ethyl]amino}propyl)-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-morpholin-4-yl[1,3]thiazolo[5,4-b]pyridin-2-yl)propanamide
N-{3-[(3,4-dichlorobenzyl)(isopropyl)]amino]propyl}-3-(5-morpholin-4-yl[1,3]thiazolo[5,4-b]pyridin-2-yl)propanamide,
N-{3-[(3,4-dichlorobenzyl)(tert-butyl)]amino]propyl}-3-(5-morpholin-4-yl[1,3]thiazolo[5,4-b]pyridin-2-yl)propanamide;
   and their salts, solvates and isomers and the salts and solvates thereof.

The present invention relates furthermore to the pharmaceutical preparations containing the compounds of the general formula (I) or its isomers, salts or solvates, which are preferably oral preparations, but inhalable, parenteral and transdermal preparation also form a subject of the present invention. The above pharmaceutical preparations may be solid or liquid formulations, for example tablets, pellets, capsules, patches, solutions, suspensions or emulsions. The solid formulations, first of all the tablets and capsules are preferred.

The above pharmaceutical preparations are prepared by applying the usual excipients and technological operations.

The compounds of the general formula (I) according to the invention can be used for the treatment of pathologies where CCR3 receptors play a role in the development of the disease.

The compounds according to the present invention can favourably used in the treatment of diseases like asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.

A further subject of the invention is the use of the compounds of the general formula (I) for the treatment of the above pathologies. The suggested daily dose is 1-100 mg of the active component, depending on the nature and severity of the disease and the sex and weight of the patient.

A further subject of the invention is the preparation of the compounds of general formula (I) where in the formula Ar¹, X, Y, Z, R¹, R² and Ar², have the meanings as defined above, and their salts, solvates and isomers.

Scheme 1. demonstrates one of the processes (version a.) for the preparation of the compounds of general formula (I).

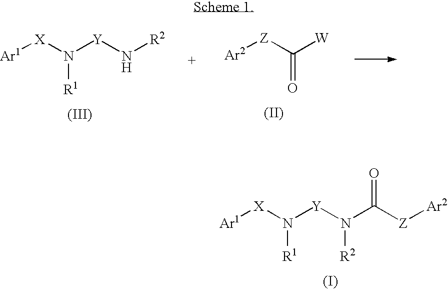

In process version a.) according to the invention a diamino-compound of general formula (III),

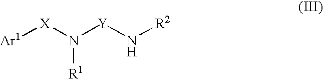

where the meanings of Ar¹, X, Y, R¹ and R² are as defined above is reacted with a carboxylic acid derivative of general formula (II),

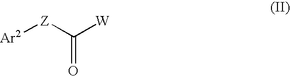

where the meanings of Ar² and Z are as defined above, W stands for halogen atom, hydroxyl group, —O(C₁₋₄alkyl)-group or —OCO-Z-Ar²-group, where Z and Ar² have the meanings as defined above, and if desired the substituents of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

In a preferred embodiment of process version a.) according to the invention, a compound of general formula (II) where W stands for hydroxyl group, is transformed with acid chloride-forming reagents, preferably with thionyl chloride, into the acid chloride, which is then reacted with the amine of general formula (III) in an inert solvent (e.g. halogenated carbohydrates, such dichloromethane, chloroform, or ethyl-acetate in the presence of a base (e.g. triethylamine) or in pyridine, at room temperature or at the reflux temperature of the reaction mixture.

A preferred method is when the acid of general formula (II) is reacted with the amine of general formula (III) in the presence of an activating agent. Activation of the carboxylic acid may take place by the preparation of mixed anhydride intermediates with the help of e.g. with pivalyl chloride (M. T. Leplawy: Tetrahedron 1960, 11, 39), ethyl chloroformate (T. Wieland: J. Liebigs Ann. Chem. 1951, 572, 190), isobutyl chloroformate (J. R. Vaughan: JACS. 1951, 73, 3547) or dicyclohexyl carbodiimide (DCC) (R. Arshady: J. Chem. Soc. Perkin Trans. 1, 1981, 529 or D. Hudson: J. Org. Chem. 1988, 53, 617), in inert solvents (e.g. dichloromethane, chloroform, tetrahydrofuran, acetonitrile), in the presence of an acid binding agent, e.g. tertiary amines (triethylamine, N-methylmorpholine), at a temperature between −10° C. and 25° C.

Activation can be achieved by using carbonyl diimidazole (H. A. Staab: Lieb. Ann. Chem.: 1957, 609, 75), in inert solvents, preferably dichloromethane, chloroform, tetrahydrofuran, acetonitrile or in the mixture thereof. Activation can also be carried out with benzotriazol-1-yl-oxy-tripyrrolidinophosphonium hexafluoro phosphate (PyBOP) in inert solvent (J. Corte: Tetrahedron Lett. 31, 1990, 205).

If the compound of general formula (II) is a carboxylic acid ester, where in the formula W stands for —O(C₁₋₄ alkyl) group, the reaction is preferably carried out at 150° C., without solvent, in melt.

The compounds of general formula (I) according to the invention can be prepared by the method shown in Scheme 2. (process version b.)

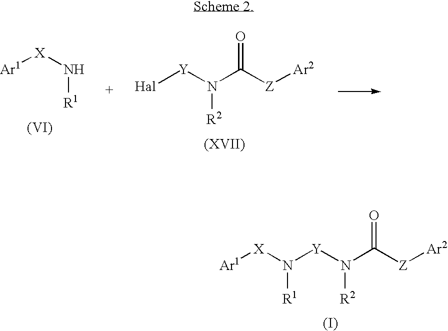

According to process version b.) the amino compound of general formula (VI),

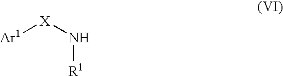

where Ar¹, X, and R¹ have the meanings as defined above, is reacted with a halogen compound of general formula (XVII),

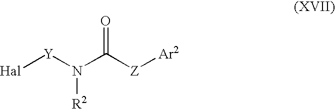

where the meanings of Y, R², Ar² and Z are as defined above and Hal means halogen atom, and if desired the substituents of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

In a preferred method of process version b.) according to the invention, the reaction of the amine of general formula (VI) and the halogen compound of general formula (XVII) is carried out in inert solvent, preferably dichloromethane, in the presence of an organic base as acid binder.

Resolution of the racemic compounds of general formula (I) to their enantiomers can be carried out by chiral preparative column chromatography, or by other methods known for the resolution of compounds of basic character.

The starting diamines of the general formula (III) may be prepared by different methods depending on the nature of the substituents R¹, R² and Y.

Scheme 3. presents the preparation of amines of the general formula (III) where R²=hydrogen atom, Y=1,3-propylene, 1-methylpropylene, 2-methylpropylene or 1,4-butylene (R⁶ and R⁷ independently represent hydrogen atom or methyl group, p is 0 or 1), and the meanings of Ar¹ and X are as defined above.

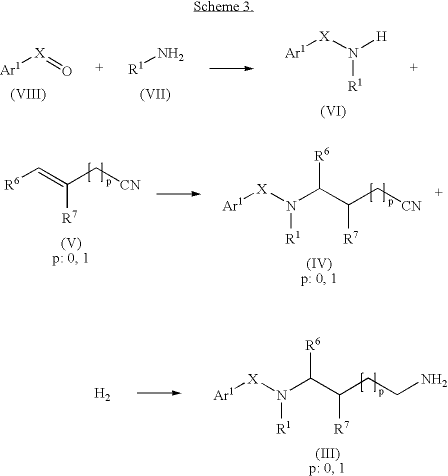

The compounds of the general formula (VI) can be prepared by methods known in the literature starting from the oxo compounds (aldehydes or ketones) of the general formula (VI) by reductive amination with the amines of general formula (VII) in alcoholic medium, in the presence of sodium cyanoborohydride (Holzgrabe U.: Arch. Pharm. 1987, 320, 7, 647-654), or by catalytic hydrogenation (Elslager E. F.: J. Med. Chem. 1981, 24, 2, 140-145), or with sodium borohydride in aqueous alcohol medium (Simig Gy.: J. Chem. Soc Perkin Trans. 1. 1992, 13, 1613-16). The compounds of the general formula (VII) are commercially available. The aldehydes of general formula (VIII) are commercially available or can be prepared by methods known in the literature. The compounds of general formula (IV) can be prepared from the amines of general formula (VI) with the alkene-cyanides of the general formula (V) by literature analogies (King M. et al: JACS. 1946, 68, 1468, or Surrey et al: JACS. 1956, 78, 2573). The cyanides of the general formula (V) are commercially available. The diamines of the general formula (III) can be obtained by catalytic hydrogenation of the cyanides of general formula (IV) by literature analogies, in alcohol or hexane solution, in the presence of ammonia and Raney nickel or rhodium catalyst, in a given case under pressure (Shapiro et al: JACS. 1959, 81, 3083-84, and Roufos I.: J. Med. Chem. 1996, 39, 7, 1514).

The amines of the general formula (III), where in the formula the meaning of Y is ethylene group, R2 stands for hydrogen atom and the meanings of Ar1 and X are as defined above, can be prepared as shown in Scheme 4.,

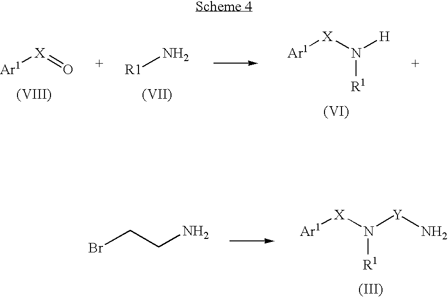

from the amines of the general formula (VI) with 2-bromoethylamine, by literature analogy, in hot aqueous solution (Arz. Forsch. 1975, 25, 1853-58).

Scheme 5. shows the preparation of the amines of general formula (III), where R2 stands for hydrogen atom, Y for 3-methylpropylene group and the meanings of Ar1 and X are as defined above,

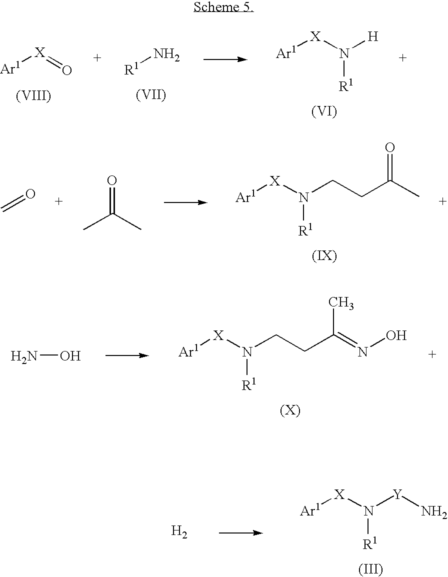

The compounds of general formula (XI) are obtained by Mannich condensation from the amines of general formula (VI) with paraformaldehyde and acetone. By literature analogy, the reaction can be performed in i-propanol under reflux conditions (JACS. 1959, 81, 2214-18). The oximes of general formula (X) are prepared from the compounds of general formula (IX) with hydroxylamine, by literature analogies, in aqueous i-propanol solution (JACS. 1959, 81, 2214-18). The amine of general formula (III) is prepared by literature analogy from the oxime of general formula (X) by catalytic hydrogenation in the presence of Raney-Nickel catalyst, in ethanolic ammonia solution.

Scheme 6. demonstrates the preparation of the amines of general formula (III) where R¹ and R² represents methyl group and the meanings of Ar¹, X and Y are as defined above.

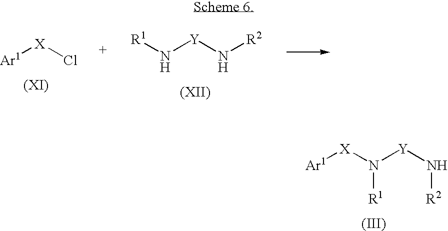

The compounds of the general formula (III) can be obtained by reacting the commercially available halogenides of the general formula (XI) with the N,N′-dimethylaminoalkyl compounds of general formula (XII), in inert solvents, preferably in acetonitrile, in the presence of an acid binding organic amine.

The oxo compounds of the general formula (VI) may be prepared by different methods depending on the nature of the X group.

The intermediate the general formula (VI), where X represents 1,3-propylene group and the meaning of Ar¹ is as defined above, can be obtained as presented in Scheme 7.,

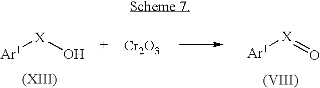

by analogies in the literature (J. Org. Chem. 2002, 67, 25, 8758-8763), from the appropriate alcohols of general formula (XIII) by oxidation with pyridinium chlorochromate in inert solvent, preferably in dichloromethane.

The intermediate of general formula (VI), where X=—CH2-CH2-CH(CH3)- and the meaning of Ar¹ is as defined above, can be prepared by the method shown in Scheme 8.,

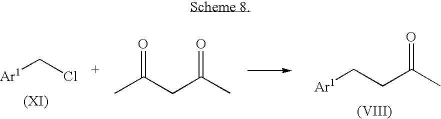

by analogies in the literature (Powel et al: JACS. 2004, 126, 25, 7788-89), by heating the commercially available benzylchlorides of general formula (XI) with pentane-2,4-dione in alcohol solution under reflux conditions, in the presence of potassium carbonate.

The carboxylic acids of general formula (II) and their esters are commercially available or they can be prepared by methods known in the literature.

The benzothiazol-2-ylpropionic acid can be synthesized from the appropriately substituted 2-mercaptoaniline with succinic acid anhydride, by heating in toluene under reflux conditions (Babitschew et al.: Ukr. Khim. Zh. 22, 1956, 211, CA 1957, 37399). The benzoxazol-2-ylpropioic acids are prepared from the appropriately substituted 2-hydroxyanilines, by analogy of the preparation of the benzothiazol-2-ylpropionic acids. The benzimidazol-2-ylpropionic acids can be obtained from the appropriately substituted 1,2-diaminobenzenes with succinic acid anhydride (Anderlini et al.: Gazz. Chim. Ital, 24, I., 1894, 141 or Lettre et al.: Chem. Ber. 84, 1951, 719). The thiazolo[5,4-d]pyrimidin-2-ylpropionic acids can be prepared from the appropriately substituted 5-aminopyrimidin-4-thioles by melting with succinic acid at high temperature (100° C.-210° C.) by literature analogies (M. Ishidate: Chem. Pharm. Bull. 8, 1960, 131). Often, the reaction takes place in two steps, in the first step only the N-(4-mercapto-5-yl)succinic acid is formed which gives the ring closured product on boiling in diluted hydrochloric acid. The thiazolo[5,4-b]pyridin-2-ylpropionic acids can be prepared by analogy with the preparation of the thiazolo[5,4-d]pyirimidin-2-ylpropionic acids, from the appropriately substituted 3-aminopyridine-2-thiol by melting with succinic acid at high temperature (100° C.-210° C.). The 3-benzoxazol-2-ylacrylic acids are prepared as described in the literature, from the appropriately substituted 2-aminophenoles by heating with maleic acid at 100° C.-210° C. (Ried et al.: Chem. Ber. 89, 1956, 2578).

The 3-[1,2,4]triazolo[1,5-a]pyridin-2-ylpropionic acid esters can be obtained as shown in Scheme 9.

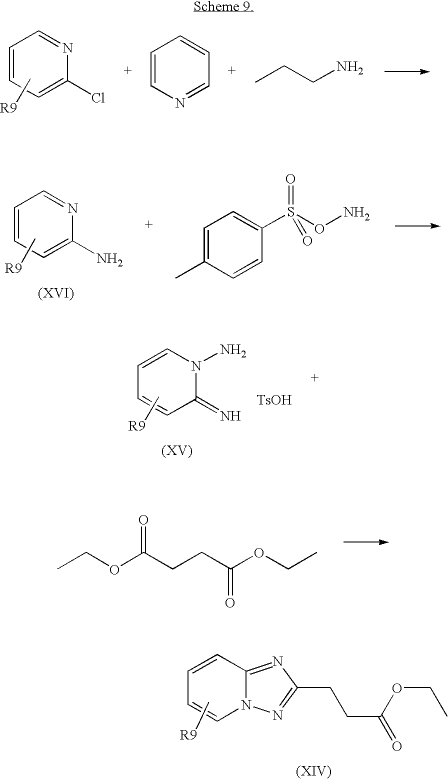

The 2-aminopyridine derivative of general formula (XVI), where R⁹ represents halogen atom or C₁₋₄ alkyl group, can be prepared from 2-chloropyridines with propylamine in the presence of pyridine chlorohydrate. This compound and o-tosylhydroxylamine results the 1-amino-2-imino-2H-pyridine tosylate of general formula (XV), which with ethyl succinate gives the 3-[1,2,4]triazolo[1,5-a]pyridin-2-ylpropionic acid esters of general formula (XV).

The compounds of general formula (IIa) forming a narrower group of the compounds of general formula (II),

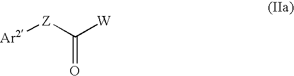

where in the formula
Ar^{2′} represents a 1,2,4-triazolo[1,5-a]pyridine- or thiazolo[5,4-b]pyridine group optionally substituted with one or more straight or branched C₁₋₄ alkyl group, straight or branched C₁₋₄ alkoxy group, hydroxyl group, —NR¹⁰R¹¹ group, —CONR¹⁰R¹¹ group, —SO₂NR¹⁰R¹¹ group, wherein the meanings of R¹⁰ and R¹¹ are as defined above;
Z represents 1,3-propylene group; and
W means as defined above; are new and also subject of the present invention.

The intermediate of general formula (XVII) can be gained by the method shown in Scheme 10.

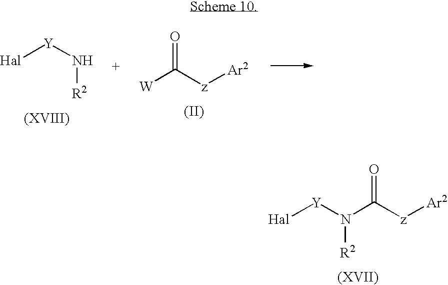

Further details of the invention are demonstrated by the following examples, without limiting the invention to the examples.

### EXAMPLE 1

### N-{3-[(3,4-Dichlorobenzyl)(methyl)amino]propyl}-3-(5-isopropylamino-thiazolo[5,4-b]pyridin-2-yl)propanamide

In the general formula (I) Ar¹ stands for 3,4-dichlorophenyl group, X for methylene group, R¹ for methyl group, R² for hydrogen atom Y for 1,3-propylene group, Z for ethylene group, Ar² for 5-1-propylamino-thiazolo[5,4-b]pyridin-2-yl group.

### a.) 3-(5-Isopropylaminothiazolo[5,4-b]pyridin-2-yl)propionic acid hydrogen chloride salt

### a/1.) N-(6-isopropylamino-2-mercaptopyridin-3-yl)succinic amide

0.5 g (2.73 mmol) 3-amino-6-isopropylaminopyridin-2-thiol is dissolved in 10 ml of toluene, under stirring 0.28 g (2.8 mmol) succinic acid anhydride is added to the solution and the mixture is heated under reflux for 1 hour. Toluene is distilled off, the residue is crystallized by treatment with ether, the crystals are filtered off and washed with ether. 0.5 g title compound is obtained in the form of an oil.

LC-MS[MH⁺]=284 (C₁₂H₁₇N₃O₃S 283.35)

### a/2.) 3-(5-Isopropylaminothiazolo[5,4-b]pyridin-2-yl)propionic acid hydrogen chloride salt

0.5 g (1.7 mmol) N-(6-isopropylamino-2-mercaptopyridin-3-yl)succinic amide is dissolved in 10 ml 10% hydrochloric acid and the solution is boiled for 10 minutes. After evaporation 0.47 g title compound is obtained in the form of an oil.

LC-MS[MH⁺]=266 (C₁₂H₁₅N₃O2S 265.34)

### b.) N-(3,4-Dichlorobenzyl)-N-(methyl)propan-1,3-diamine

20 g (82.3 mmol) 3-[(3,4-Dichlorobenzyl)(methyl)amino]propionitrile is hydrogenated at room temperature, in the presence of Raney-Nickel catalyst, in ethanolic ammonia solution in (100 ml). After removal of the solvent 20 g title compound is obtained in the form of an oil. LC/MS[MH⁺]=247 (C₁₁H₁₆Cl₂N₂ 247.17)

### c.) N-{3-[(3,4-Dichlorobenzyl)(methyl)amino]propyl}-3-(5-isopropylamino-thiazolo[5,4-b]pyridin-2-yl)propanamide

0.28 g (0.93 mmol) 3-(5-Isopropylaminothiazolo[5,4-b]pyridin-2-yl)propionic acid hydrogen chloride salt is dissolved in 8 ml dry dimethylformamide, 0.18 g (1.12 mmol) N,N-carbonyl-diimidazole is added to it, the mixture is stirred for 1 hour at room temperature, then 0.23 g (0.96 mmol) N-(3,4-dichlorobenzyl)-N-(methyl)propan-1,3-diamine in 1 ml dimethylformamide is added dropwise and stirring is continued for 2 hours. The reaction mixture is poured onto ice-water and alkalinized with 1N sodium hydroxide solution, extracted with 3×10 ml ether, the combined ether phase is washed with water, dried over sodium sulfate, evaporated in vacuum, and purified by column chromatography using chloroform-methanol 100:1, 100:2 and 100:5 mixtures. 100 mg title compound is obtained in the form of an oil. LC-MS[MH⁺]=494 (C₂₃H₂₉Cl₂N₅OS 494,49)

### EXAMPLES 2-21

The compounds of Table 1. are prepared according to the method described in Example 1.

TABLE 1

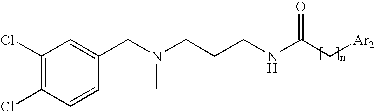

| Example | n | Ar² | Mp (° C.) | [MH⁺] |
|---|---|---|---|---|
| 2. | 2 | 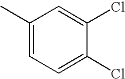 | | [MH⁺] = 447 |
| 3. | 2 | 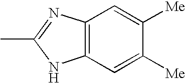 | | [MH⁺] = 447 |
| 4. | 2 | 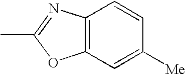 | | [MH⁺] = 434 |
| 5. | 2 | 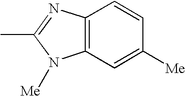 | | [MH⁺] = 447 |
| 6. | 2 | 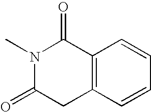 | | [MH⁺] = 462 |
| 7. | 2 | 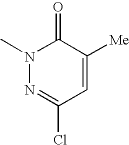 | 61.5-63° C. | |
| 8. | 2 | 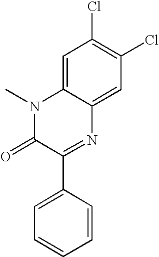 | 157° C. | |
| 9. | 2 | 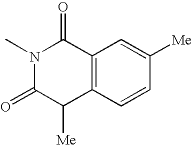 | 89-93° C. | |
| 10. | 2 | 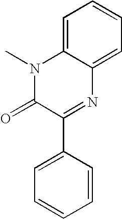 | 76.5-83° C. | |
| 11. | 3 | 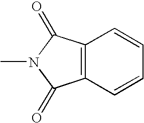 | 83.5-84.5° C. | |
| 12. | 2 | 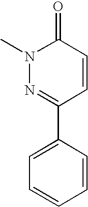 | | [MH⁺] = 473 |
| 13. | 2(CH═CH) | 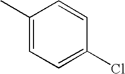 | 66-68° C. | |
| 14. | 2 | 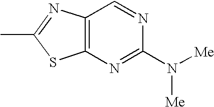 | | [MH⁺] = 481 |
| 15. | 2 | 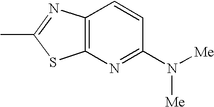 | 68-69° C. | |
| 16. | 2 | 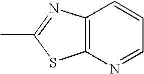 | | [MH⁺] = 437 |
| 17. | 2(CH═CH) | 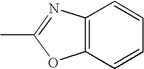 | 34-36° C. | |
| 18. | 2 | 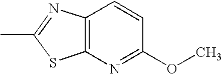 | 69-70° C. | |
| 19. | 2 | 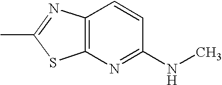 | 104-105° C. | |
| 20. | 2 | 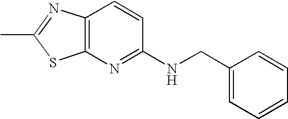 | | [MH⁺] = 560 |
| 21. | 2 | 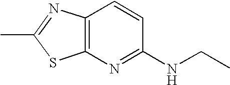 | | [MH⁺] = 480 |

### EXAMPLE 22

### N-{3-[(3,4-dichlorobenzyl)(methyl)amino]propyl}-3-phenylpropanamide

In the general formula (I) Ar¹ stands for 3,4-dichlorophenyl group, X for methylene group, R¹ for methyl group, R² for hydrogen atom Y for 1,3-propylene group, Z for ethylene group, Ar² for phenyl group.

### a.) N-(3-Bromopropyl)-3-phenylpropanamide

0.44 g (2 mmol) 3-bromopropylamine hydrogen bromide salt is dissolved in the solution of 0.16 g (4 mmol) sodium hydroxide in 4 ml of water and under ice-water cooling 0.34 g (2 mmol) phenylpropionyl chloride is added. The mixture is stirred for 1 hour under cooling and 5 hours at room temperature. The resulting crystals are filtered off and washed with water to obtain the title compound. LC-MS[MH⁺]=271 (C₁₂H₁₆BrNO 270.17)

### b.) N-{3-[(3,4-dichlorobenzyl)(methyl)amino]propyl}-3-phenylpropanamide

To the solution of 0.28 g (1.5 mmol) (3,4-dichlorobenzyl)(methyl)amine in 3 ml dichloromethane 0.2 ml (1.5 mmol) triethylamine is added and the solution of 0.4 g (1.5 mmol) N-(3-bromopropyl)-3-phenylpropionamide in 3 ml of dichloromethane is added to it dropwise. The mixture is stirred at room temperature for 4 hours. The solvent is removed, to the residue water and ethyl acetate are added and the mixture is extracted with 3×15 ml ethyl acetate. The organic phase is washed with water, dried over sodium sulfate and evaporated in vacuum to obtain the title compound.

LC-MS[MH⁺]=379 (C₂₀H₂₄Cl₂N₂O 379.33)

### EXAMPLE 23

### 3-Benzothiazol-2-yl-N-{3-[(3,4-dichlorobenzyl)(methyl)amino]propyl}propanamide

In the general formula (I) Ar¹ stands for 3,4-dichlorophenyl group, X for methylene group, R¹ for methyl group, R² for hydrogen atom Y for 1,3-propylene group, Z for ethylene group, Ar² for benzothiazol-2-yl group.

0.2 g (1 mmol) 3-benzothiazol-2-ylpropionic acid is dissolved in 5 ml chloroform and 0.11 ml (1 mmol) N-methylmorpholine is added to it. The mixture is cooled to −10° C., 0.095 ml (1 mmol) ethyl chloroformate and after 15 minutes of stirring 0.3 g (1.2 mM) N-(3,4-dichlorobenzyl)-N-(methyl)propane-1,3-diamine in 3 ml chloroform are added to the mixture. Stirring is continued for 0.5 hour under cooling and 0.5 hour at room temperature. The solution is washed with water, then with 5% potassium hydrogen sulfate solution, dried over sodium sulfate, evaporated in vacuum and purified by column chromatography to obtain 70 mg title compound is obtained in the form of an oil. LC-MS[MH⁺]=436 (C₂₁H₂₃Cl₂N₃OS 436,41).

### EXAMPLES 24-26

The compounds of Table 2. are prepared according to the method described in Example 23.

TABLE 2

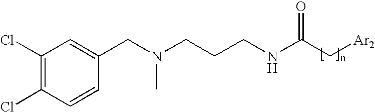

| Ex- | | | Mp | |
|---|---|---|---|---|
| ample | n | Ar² | (° C.) | [MH⁺] |
| 24. | 2 | 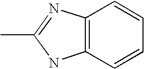 | 120-122° C. | |
| 25. | 2 | 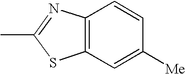 | 159-161° C. | |
| 26. | 2(CH═CH) | 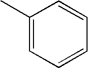 | 192-194° C. | |

### EXAMPLE 27

### N-{3-[3,4-Dichlorobenzyl)(methyl)amino]propyl}-3-(7-ethylamino-[1,2,4]triazolo[1,5-a]pyridin-2-yl)propanamide

In the general formula (D) Ar¹ stands for 3,4-dichlorophenyl group, X for methylene group, R¹ for methyl group, R² for hydrogen atom Y for 1,3-propylene group, Z for ethylene group, Ar² for 3-(7-ethylamino-[1,2,4]triazolo[1,5-a]pyridin-2-yl) group.

### a.) (2-Chloropyridin-4-yl)(ethyl)-amine

To the solution of 5.7 g (36 mmol) 2-chloro-4-nitropyridine in 100 ml ethanol 11.8 ml (180 mmol) ethylamine is added. The reaction mixture is stirred at room temperature for 24 hours, evaporated, to the residue 10 ml 2 N sodium hydroxide solution and 10 ml of water are added and the mixture is extracted with 2×15 ml dichloromethane. The organic phase is dried over sodium sulfate and evaporated in vacuum to obtain 5.5 g title compound as crystals. Mp: 55-57° C.

### b.) (2-Aminopyridin-4-yl)(ethyl)amine

To the solution of 5.3 g (34 mmol) (2-chloropyridin-4-yl)(ethyl)amine in 75 ml pyridine, 28 ml 25% hydrogen chloride in ether solution is dropped. After heating the solution under reflux for 80 hours, 22.4 ml propylamine is added and heating is continued for 2.5 hours. The solvent is removed, to the residue 25 ml 40% sodium hydroxide solution and 25 ml ethanol are added, the precipitated crystalline material is filtered off, washed with ethanol. The mother liquor is evaporated, the residual oil is purified by column chromatography using ethyl acetate-methanol-ammonium hydroxide 250:20:5 mixture as eluent. 3.8 g title compound is obtained in the form of an oil. LC-MS[MH⁺]=138 (C₇H₁₁N₃ 137.185).

### c.) N⁴-Ethyl-2-iminopyridin-1,4(2H)-diamine tosylate

The solution of 5.8 g (31.2 mmol) O-tosyl-hydroxylamine in 100 ml dichloromethane is dropped under ice-water cooling to the solution of 3.6 g (26 mmol) (2-aminopyridin-4-yl)(ethyl)amine in 25 ml dichloromethane. The reaction mixture is stirred for 30 minutes under cooling and 2 hours at room temperature. The precipitate is filtered off, washed with dichloromethane. 4.9 g title compound is obtained. Mp.: 220-222° C.

### d.) Ethyl 3-(7-ethylamino-[1,2,4]triazolo)[1,5-a]pyridin-2-yl]propionate

To the suspension of 4.2 g (13 mmol) N⁴-Ethyl-2-iminopyridin-1,4(2B)-diamine tosylate in 65 ml ethanol, 9 g (65 mmol) water-free potassium carbonate and 10.8 ml (65 mmol) ethyl succinate are added. The reaction mixture is heated under reflux for 8 hours, then 130 ml water is added and the mixture is extracted with 3×40 ml dichloromethane. The united organic phase is dried over sodium sulfate and evaporated in vacuum. To the residual oil 100 ml petrolether is added, the precipitated crystals are filtered off and purified by column chromatography. The resulting oily material is crystallized in petrolether-ether 9:1 mixture, the crystals are filtered off. 1.17 g title compound is obtained. Mp.: 147-149° C.

### e.) N-{3-[3,4-Dichlorobenzyl)(methyl)amino]propyl}-3-(7-ethylamino[1,2,4]triazolo[1,5-a]pyridin-2-yl)propanamide

The mixture of 0.52 g (2 mmol) ethyl 3-(7-ethylamino[1,2,4]triazolo[1,5-a]pyridin-2-yl-propionate and 0.5 g (2 mmol) N-(3,4-dichlorobenzyl)-N-(methyl)propane-1,3-diamine is heated at 100° C. for 42 hours. After cooling, the resulted oil is purified by column chromatography using chloroform-methanol mixture as eluent. 95 mg title compound is obtained in the form of an oil. LC-MS[MH⁺]=463 (C₂₂H₂₈Cl₂N6O 463.410).

### EXAMPLES 28-35

The compounds of Table 3. are prepared according to the method described in Example 27.

TABLE 3

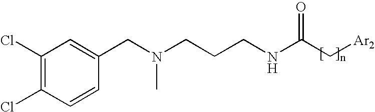

| Ex- | | | Mp | |
|---|---|---|---|---|
| ample | n | Ar2 | (° C.) | [MH⁺] |
| 28. | 2 | 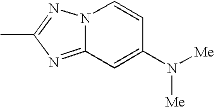 | | [MH⁺] = 463 |
| 29. | 2 | 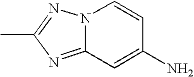 | 125-127° C. | |
| 30. | 2 | 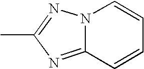 | | [MH⁺] = 420 |
| 31. | 3 | 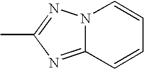 | | [MH⁺] = 434 |
| 32. | 3 | 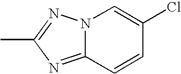 | | [MH⁺] = 414 |
| 33. | 2 | 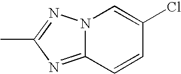 | 113-114° C. | |
| 34. | 3 | 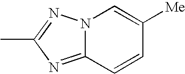 | | [MH⁺] = 448 |
| 35. | 3 | 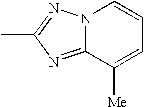 | | [MH⁺] = 448 |

### EXAMPLE 36

### N-(3-{[1-(3,4-Dichlorophenyl)ethyl]amino}propyl)-3-(5-methylamino[1,3]thiazolo[5,4-b]pyridin-2-yl)propanamide

In the general formula (I) Ar¹ stands for 3,4-dichlorophenyl group, X for —CH(CH₃)— group, R¹ for hydrogen atom, R² for hydrogen atom, Y for 1,3-propylene group, Z for ethylene group, Ar² for 5-methylamino[1,3]thiazolo[5,4-b]pyridin-2-yl group

### a.) 3-(5-Methylamino[1,3]thiazolo[5,4-b]pyridin-2-yl)propionic acid hydrogen chloride salt

According to the procedure described in Example 1.a.) starting from 3.76 g (24.22 mmol) 3-amino-6-methylaminopyridin-2-thiol, 4.9 g title compound is obtained. Mp: 202-204° C.

### b.) N-[1-(3,4-Dichlorophenyl)ethyl]-propan-1,3-diamine

### b/1.) [1-(3,4-dichlorophenyl)ethyl]amine

To the solution of 5 g (26.45 mmol) 3,4-dichloro-acetophenon in 66 ml methanol 25.4 g (0.33 mol) ammonium acetate and 1.2 g (19.1 mmol) sodium-cyano-borohydride are added under stirring at room temperature and stirring is continued for 24 hours. The reaction mixture is poured to 15 ml 5N hydrochloric acid solution under ice-water cooling then extracted with 2×15 ml ether. The acidic solution is alkalinized to pH 9, the aqueous solution is extracted with 3×20 ml dichloromethane, dried over sodium sulfate, filtered off, evaporated in vacuum. Thus 2.7 g title compound is obtained in the form of an oil.

LC-MS[MH⁺]=190 (C₈H₉Cl₂N 190.072).

### b/2.) 3′-{[1-(3,4-Dichlorophenyl)ethyl]amino}propionitrile

To the solution of 1.1 g (5.8 mmol) [1-(3,4-dichlorophenyl)ethyl]amine in 11 ml abs. methanol 0.4 ml (6 mmol) acrylonitrile is added under ice-water cooling, then the stirring is continued for 24 hours at room temperature. The solution is evaporated in vacuum to obtain 1.2 g title compound in the form of an oil.

LC-MS[MH⁺]=243 (C₁₁H₁₂Cl₂N₂ 243.136).

### b.) N-[1-(3,4-Dichlorophenyl)ethyl]-propan-1,3-diamine

To the solution of 1.2 g (4.94 mmol) 3′-{[1-(3,4-dichlorophenyl)ethyl]amino}propionitrile in 20 ml methanol 10 ml 25% ammonium hydroxide solution is added and hydrogenated in the presence of Raney-Nickel catalyst under 30 bar pressure at room temperature then at 35° C. The solution is evaporated in vacuum to obtain 1.1 g title compound in the form of an oil. LC-MS[MH⁺]=247 (C₁₁H₁₆Cl₂N₂ 247.167).

### c.) N-(3-{[1-(3,4-Dichlorophenyl)ethyl]amino}propyl)-3-(5-methylamino[1,3]thiazolo[5,4-b]pyridin-2-yl)propanamide

0.5 g (2.02 mmol) 3-[5-(methylamino)[1,3]thiazolo[5,4-b]pyridin-2-yl)propionic acid hydrogen chloride salt is dissolved in 15 ml anhydrous dimethylformamide and 0.35 g (2.16 mmol) N,N-carbonyldiimidazole and 0.3 ml (2.15 mmol) triethylamine are added to the solution and stirred for 1 hour at room temperature. Then the solution of 0.55 g (2.01 mmol) N-[1-(3,4-dichlorophenyl)ethyl]-propan-1,3-diamine in 5 ml dimethylformamide is added dropwise and stirred for further 2 hours. The reaction mixture is poured onto ice-water and alkalinized with 1N sodium-hydroxide solution, then extracted with 3×10 ml ether, the united ether solution is washed with water, dried over sodium sulfate evaporated in vacuum and purified by column chromatography using chloroform-methanol 100:1, 100:2, 100:5 mixtures with increasing polarity, as eluent. Thus 100 mg title compound is obtained in the form of an oil. LC-MS[MH⁺]=466 (C₂₁H₂₅Cl₂N₅OS 466,435).

### EXAMPLE 37

### N-{3-[[1-(3,4-Dichlorophenyl)ethyl])(methyl)amino]propyl}-3-(5-methylamino[1,3]thiazolo[5,4-b]pyridin-2-yl)propanamide

In the general formula (I) Ar¹ stands for 3,4-dichlorophenyl group, X for —CH(CH₃)— group, R¹ for methyl group, R² for hydrogen atom Y for 1,3-propylene group, Z for ethylene group, Ar² for 5-methylamino[1,3]thiazolo[5,4-b]pyridin-2-yl group.

### a.) 3-(5-Methylamino[1,3]thiazolo[5,4-b]pyridin-2-yl)propionic acid hydrogen chloride salt

According to the procedure described in Example 1.a.) starting from 3.76 g (24.22 mmol) 3-amino-6-methylaminopyridin-2-thiol, 4.9 g title compound is obtained. Mp: 202-204° C.

### b.) N-[1-(3,4-dichlorophenyl)ethyl]-N-methylpropan-1,3-diamine

### b/1.) [1-(3,4-dichlorophenyl)ethyl]methylamine

40 ml ethanol and 6.4 ml 25% solution of hydrochloric acid in ethanol are added to 16 ml 33% solution of methylamine in ethanol then 4 g (21.16 mmol) 3,4-dichloroacetophenone is added at room temperature under stirring. 2.64 g (42 mmol) sodium cyanoborohydride is added under cooling and stirred for 24 hours. The precipitated crystals are filtered off, the ethanolic mother liquor is evaporated in vacuum, after the addition of water the reaction mixture is acidified with 2N hydrochloric acid solution to pH 3 then extracted with 2×15 ml ether. The acidic solution is alkalinized to pH 9, the aqueous solution is extracted with 3×20 ml dichloromethane, dried over sodium sulfate, filtered off, evaporated in vacuum to obtain 3.3 g title compound in the form of an oil.

LC-MS[MH⁺]=204 (C₉H1₁Cl₂N 204,099).

### b/2.) 3-[[1-(3,4-Dichlorophenyl)ethyl](methyl)amino]propionitrile

To the solution of 3.3 g (16.2 mmol) [1-(3,4-dichlorophenyl)ethyl]methylamine in 33 ml abs. methanol 1.1 ml (16.7 mmol) acrylonitrile is added under ice-water cooling, then stirring is continued at room temperature for 24 hours. The solution is evaporated in vacuum to obtain 3.9 g title compound in the form of an oil.

LC-MS[MH⁺]=257 (C₁₂H₁₄Cl₂N₂ 257,163).

### b.) N-[1-(3,4-Dichlorophenyl)ethyl]-N-methylpropan-1,3-diamine

To the solution of 1.9 g (7.4 mmol) 3-[[1-(3,4-dichlorophenyl)ethyl](methyl)amino]propionitrile in 20 ml methanol 20 ml 25% ammonium hydroxide solution is added and hydrogenated in the presence of Raney-Nickel catalyst under 30 bar pressure at 45° C. The solution is evaporated in vacuum to obtain 1.9 g title compound in the form of an oil.

LC-MS[MH⁺]=261 (C₁₂H₁₈Cl₂N₂ 261,2).

### c.) N-{3-[[1-(3,4-Dichlorophenyl)ethyl])(methyl)amino]propyl}-3-(5-methylamino[1,3]thiazolo[5,4-b]pyridin-2-yl)propanamide

0.5 g (1.91 mmol) 3-(5-methylaminothiazolo[5,4-b]pyridin-2-yl)propionic acid hydrogen chloride salt is dissolved in 10 ml anhydrous dimethylformamide and 0.34 g (2.1 mmol) N,N-carbonyldiimidazole is added and stirred at room temperature for 1 hour. Then the solution of 0.52 g (1.9 mmol) N-[1-(3,4-dichlorophenyl)ethyl]-N-methylpropan-1,3-diamine in 15 ml dimethylformamide and 0.3 ml (2.15 mmol) triethylamine are added and the stirring is continued for 2 hours. The reaction mixture is poured onto ice-water and alkalinized with 1N sodium-hydroxide solution, then extracted with 3×10 ml ether, the united ether solution is washed with water, dried over sodium sulfate, evaporated in vacuum and purified by column chromatography using chloroform-methanol 100:1, 100:2, 100:5 mixtures with increasing polarity, as eluent. Thus 100 mg title compound is obtained in the form of an oil.

LC-MS[MH⁺]=480 (C₂₂H₂₇Cl₂N₅OS 480,461).

### EXAMPLE 38

### N-{3-[(3,4-Dichlorophenyl)ethyl](methyl)amino]propyl}-3-(5-cyclopropylamino[1,3]thiazolo[5,4-b)pyridin-2-yl)propanamide

In the general formula (I) Ar¹ stands for 3,4-dichlorophenyl group, X for —CH(CH₃)— group, R¹ for methyl group, R² for hydrogen atom Y for 1,3-propylene group, Z for ethylene group, Ar² for 5-cyclopropylamino[1,3]thiazolo[5,4-b]pyridin-2-yl group.

### a.) 3-(5-cyclopropylamino[1,3]thiazolo[5,4-b]pyridin-2-yl)propionic acid hydrogen chloride salt

According to the procedure described in Example 1.a.) starting from 0.2 g (1.1 mmol) 3-amino-6-cyclopropylaminopyridin-2-thiol, 0.2 g title compound is obtained.

Mp: 198-200° C.

### b.) N-{3-[(3,4-dichlorophenyl)ethyl](methyl)amino]propyl}-3-(5-cyclopropylamino[1,3]-thiazolo[5,4-b]pyridin-2-yl)propanamide

According to the procedure described in Example 37. starting from 0.22 g (0.67 mmol) 3-(5-cyclopropylamino[1,3]thiazolo-[5,4-b]pyridin-2-yl)propionic acid hydrogen chloride salt and reacting it with 0.18 g (0.69 mmol) N-[1-(3,4-dichlorophenyl)ethyl]-N-methylpropan-1,3-diamine, 50 mg title compound is obtained as white crystals.

Mp: 150-152° C.

### EXAMPLE 39

### N-{3-[[1-(3,4-Dichlorophenyl)ethyl])(methyl)amino]propyl}-3-(5-piperidin-1-yl[1,3]thiazolo[5,4-b]pyridin-2-yl)propanamide

In the general formula (I) Ar¹ stands for 3,4-dichlorophenyl group, X for —CH(CH₃)— group, R¹ for methyl group, R² for hydrogen atom Y for 1,3-propylene group, Z for ethylene group, Ar² for 5-piperidin-1-yl[1,3]thiazolo[5,4-b]pyridin-2-yl group.

### a.) 3-(5-Piperidin-1-yl[1,3]thiazolo[5,4-b]pyridin-2-yl)propionic acid hydrogen chloride salt

### a/1.) N-(4-piperidin-1-yl-2-mercaptopyridin-3-yl)succinic amide

0.5 g (2.39 mmol) 3-amino-6-piperidin-1-ylpyridin-2-thiol is dissolved in 15 ml toluene and 0.24 g (2.4 mmol) succinic acid anhydride is added under stirring and boiled for 1 hour. The toluene is distilled off, residue is crystallized with ether, filtered off, washed with ether. Thus 0.7 g title compound is obtained, which is used in the following reaction without drying.

LC-MS[MH⁺]=292 (C₁₄H₁₇N₃O₂S 291,35)

### a.) 3-(5-Piperidin-1-yl[1,3]thiazolo[5,4-b]pyridin-2-yl)propionic acid hydrogen chloride salt

0.7 g N-(4-piperidin-1-yl-2-mercaptopyridin-3-yl)succinic amide is dissolved in 17 ml 10% hydrochloric acid and the solution is boiled for 45 minutes. The precipitated crystalline product is filtered off, washed with water to obtain 0.62 g title compound.

Mp: 214-216° C.

### b.) N-{3-[[1-(3,4-dichlorophenyl)ethyl])(methyl)amino]propyl}-3-(5-piperidin-1-yl[1,3]thiazolo[5,4-b]pyridin-2-yl)propanamide

0.4 g (1.22 mmol) 3-(5-piperidin-1-yl[1,3]thiazolo[5,4-b]pyridin-2-yl)propionic acid hydrogen chloride salt is dissolved in 10 ml anhydrous dimethylformamide and 0.24 g (1.48 mmol) N,N-carbonyldiimidazole is added and stirred for 1 hour at room temperature. Then the solution of 0.34 g (1.3 mmol) N-[1-(3,4-dichlorophenyl)ethyl]-N-methylpropan-1,3-diamine (prepared according to Example 37.) in 6 ml dimethylformamide, which contains 0.42 ml (3 mmol) triethylamine, is added dropwise and the stirring is continued for 24 hours. The reaction mixture is poured onto ice-water and alkalinized with 1N sodium-hydroxide solution, then extracted with 3×10 ml ether, the united ether solution is washed with water, dried over sodium sulfate, evaporated in vacuum and purified by column chromatography using chloroform-methanol 98:2 mixture as eluent. Thus 0.21 mg title compound is obtained in the form of an oil.

LC-MS[MH⁺]=534 (C₂₆H₃₃Cl₂N₅OS 534,553).

### EXAMPLE 40

### N-{3-[[1-(3,4-Dichlorophenyl)ethyl])(methyl)amino]propyl}-3-(5-pyrrolidin-1-yl[1,3]thiazolo[5,4-b]pyridin-2-yl)propanamide

In the general formula (I) Ar¹ stands for 3,4-dichlorophenyl group, X for —CH(CH₃)— group, R¹ for methyl group, R² for hydrogen atom Y for 1,3-propylene group, Z for ethylene group, A² for 5-pyrrolidin-1-yl[1,3]thiazolo[5,4-b]pyridin-2-yl group.

### a.) 3-(5-Pyrrolidin-1-yl[1,3]thiazolo[5,4-b]pyridin-2-yl)propionic acid hydrogen chloride salt

According to the method described in Example 39. starting from 1.6 g (7.36 mmol) 3-amino-6-pyrrolidin-1-ylpyridin-2-thiol 2 g title compound is obtained as crystals.

Mp: 258-259° C.

### b.) N-{3-[[1-(3,4-Dichlorophenyl)ethyl])(methyl)amino]propyl}-3-(5-pyrrolidin-1-yl[1,3]thiazolo[5,4-b]pyridin-2-yl)propanamide

According to the method described in Example 39. starting form 0.4 g (1.27 mmol) 3-(5-pyrrolidin-1-yl[1,3]thiazolo[5,4-b]pyridin-2-yl)propionic acid hydrogen chloride salt and 0.3 g (1.15 mmol) N-[1-(3,4-dichlorophenyl)ethyl]-N-methylpropan-1,3-diamine, 0.2 g title compound is obtained in the form of an oil.

LC-MS[MH⁺]=520 (C₂₅H₃₁Cl₂N₅OS 520,526).

### EXAMPLE 41

### N-(3-{[1-(3,4-Dichlorophenyl)ethyl]amino}propyl)-3-(5-piperidin-1-yl[1,3]thiazolo[5,4-b]pyridin-2-yl)propanamide

In the general formula (I) Ar¹ stands for 3,4-dichlorophenyl group, X for —CH(CH₃)— group, R¹ for hydrogen atom, R² for hydrogen atom Y for 1,3-propylene group, Z for ethylene group, Ar² for 5-piperidin-1-yl[1,3]thiazolo[5,4-b]pyridin-2-yl group. 0.44 g (1.22 mmol) 3-(5-piperidin-1-yl[1,3]thiazolo[5,4-b]pyridin-2-yl)propionic acid hydrogen chloride salt is dissolved in 10 ml anhydrous dimethylformamide and 0.24 g (1.48 mmol) N,N-carbonyldiimidazole is added and stirred for 1 hour at room temperature. Then the solution of 0.3 g (1.23 mmol) N-[1-(3,4-dichlorophenyl)ethyl]-propan-1,3-diamine (prepared according to Example 36.) in 6 ml dimethylformamide, containing 0.4 ml (2.87 mmol) triethylamine, is added dropwise and the stirring is continued for 24 hours. The reaction mixture is poured onto ice-water and alkalinized with 1N sodium-hydroxide then extracted with 3×10 ml ether, the united ether solution is washed with water, dried over sodium sulfate, evaporated in vacuum and purified by column chromatography. Thus 0.13 g title compound is obtained in the form of an oil.

LC-MS[MH⁺]=520 (C₂₅H₃₁Cl₂N₅OS 520,526).

### EXAMPLE 42

### N-(3-{[1-(3,4-Dichlorophenyl)ethyl]amino}propyl)-3-(5-pyrrolidin-1-yl[1,3]thiazolo[5,4-b]pyridin-2-yl)propanamide

In the general formula (I) Ar¹ stands for 3,4-dichlorophenyl group, X for —CH(CH₃)— group, R¹ for hydrogen atom, R² for hydrogen atom Y for 1,3-propylene group, Z for ethylene group, Ar² for 5-pyrrolidin-1-yl[1,3]thiazolo[5,4-b]pyridin-2-yl group. According to the procedure described in Example 41. starting from 0.4 g (1.27 mmol) 3-(5-pyrrolidin-1-yl[1,3]thiazolo[5,4-b]pyridin-2-yl)propionic acid hydrogen chloride salt and 0.3 g (1.21 mmol) N-[1-(3,4-dichlorophenyl)ethyl]-propan-1,3-diamine, 0.13 g title compound is obtained in the form of an oil.

LC-MS[MH⁺]=506 (C₂₄H₂₉Cl₂N₅OS 506,449).

### EXAMPLE 43

### N-(3-{[1-(3,4-Dichlorophenyl)ethyl]amino}propyl)-3-(5-morpholin-4-yl[1,3]thiazolo[5,4-b]pyridin-2-yl)propanamide

In the general formula (I) Ar¹ stands for 3,4-dichlorophenyl group, X for —CH(CH₃)— group, R¹ for hydrogen atom, R² for hydrogen atom Y for 1,3-propylene group, Z for ethylene group, Ar² for 5-morpholin-4-yl[1,3]thiazolo[5,4-b]pyridin-2-yl group.

According to the procedure described in Example 41. starting from 0.36 g (1 mmol) 3-(5-morpholin-4-yl[1,3]thiazolo[5,4-b]pyridin-2-yl)propionic acid hydrogen chloride salt and 0.24 g (1 mmol) N-[1-(3,4-dichlorophenyl)ethyl]-propan-1,3-diamine, 45 mg title compound is obtained in the form of an oil.

LC-MS[MH⁺]=522 (C₂₄H₂₉Cl₂N₅O₂S 522,498).

### EXAMPLE 44

### N-{3-[(3,4-Dichlorobenzyl)(isopropyl)]amino]propyl}-3-(5-morpholin-4-yl[1,3]thiazolo[5,4-b]pyridin-2-yl)propanamide

In the general formula (I) Ar¹ stands for 3,4-dichlorophenyl group, X for methylene group, R¹ for isopropyl group, R² for hydrogen atom Y for 1,3-propylene group, Z for ethylene group, Ar² for 5-morpholin-4-yl[1,3]thiazolo[5,4-b]pyridin-2-yl group.

### a.) N-(3,4-dichlorobenzyl)-N-isopropylpropan-1,3-diamine

### a/1.) (3,4-Dichlorobenzyl)isopropilamine

2 g (11.43 mmol) 3,4-dichlorobenzaldehyde is dissolved in 7 ml methanol and 1.3 g (22.86 mmol) isopropylamine is added under stirring at room temperature. The reaction mixture is heated to 0° C. and 0.22 g (5.8 mmol) sodium borohydride is added to it in parts while keeping the temperature at 0° C. After the addition stirring is continued at room temperature for 2 hours. The methanol is evaporated, to the residue 8 ml water is added and extracted with 3×20 ml dichloromethane. The organic phase is washed with 10 ml water, dried over sodium sulfate, filtered off, evaporated in vacuum. After purification by column chromatography 0.96 g title compound is obtained in the form of an oil.

LC-MS[MH⁺]=218 (C₁₀H₁₃Cl₂N 218.126).

### a/2.) 3-[1-(3,4-Dichlorobenzyl)](isopropyl)amino]propionitrile

To the solution of 0.2 g (0.92 mmol) (3,4-dichlorobenzyl)isopropylamine in 1 ml abs methanol 0.09 ml (1.38 mmol) acrylonitrile is added under ice-water cooling, then the stirring is continued for 48 hours at room temperature. After evaporation in vacuum 0.28 g title compound is obtained in the form of an oil.

LC-MS[MH⁺]=271 (C₁₃H₁₆Cl₂N₂ 271,189).

### a.) N-(3,4-dichlorobenzyl)-N-isopropylpropan-1,3-diamine

To the solution of 0.25 g (0.91 mmol) 3-[1-(3,4-dichlorobenzyl)](isopropyl)amino]propionitrile in 144 ml methanol 36 ml 25% ammonium hydroxide solution is added and hydrogenated in the presence of Raney-Nickel catalyst under 30 bar pressure in a H-CUBE THALES apparatus at 45° C. The solution is evaporated in vacuum and thus 0.28 g title compound is obtained in the form of an oil.

LC-MS[MH⁺]=275 (C₁₃H₂₀Cl₂N₂ 275.221).

### b.) N-{3-[1-(3,4-Dichlorobenzyl)(isopropyl)]amino]propyl}-3-(5-morpholin-4-yl[1,3]thiazolo[5,4-b]pyridin-2-yl)propanamide

0.3 g (0.91 mmol) 3-(5-morpholin-4-yl[1,3]thiazolo[5,4-b]pyridin-2-yl)propionic acid hydrogen chloride salt is dissolved in 7 ml anhydrous dimethylformamide and 0.24 g (1.37 mmol) N,N-carbonyldiimidazole is added and stirred for 1 hour at room temperature. Then the solution of 0.25 g (0.91 mmol) N-(3,4-dichlorobenzyl)-N-isopropylpropan-1,3-diamine in 3 ml dimethylformamide and 0.25 ml (1.82 mmol) triethylamine is added dropwise and the stirring is continued for further 2 hours. The reaction mixture is poured onto ice-water and alkalinized with 1N sodium hydroxide then extracted with 3×10 ml ether. The united ether solution is washed with water, dried over sodium sulfate evaporated in vacuum and purified by column chromatography with chloroform. Thus 140 mg title compound is obtained in the form of an oil.

LC-MS[MH⁺]=550 (C₂₆H₃₃Cl₂N₅O₂S 550.552).

### EXAMPLE 45

### N-{3-[(3,4-Dichlorobenzyl)(tert-butyl)]amino]propyl}-3-(5-morpholin-4-yl[1,3]thiazolo[5,4-b]pyridin-2-yl)propanamide

In the general formula (I) Ar¹ stands for 3,4-dichlorophenyl group, X for methylene group, R¹ for tert-butyl group, R² for hydrogen atom Y for 1,3-propylene group, Z for ethylene group, Ar² for 5-mopholin-4-yl[1,3]thiazolo[5,4-b]pyridin-2-yl group.

### a.) N-(3,4-dichlorobenzyl)-N-(tert.-butyl)propan-1,3-diamine

### a/1.) N-(3,4-Dichlorobenzyl)-2-methylpropan-2-amine

According to the method described in Example 44. starting from 2 g (11.43 mmol) 3,4-dichlorobenzaldehyde reacting it with 2.4 ml (22.86 mmol) tert.-butylamine, 1.63 g title compound is obtained in the form of an oil.

LC-MS[MH⁺]=232 (C₁₁H₁₅Cl₂N 232.152).

### a/2.) 3-[1-(3,4-Dichlorobenzyl)](tert-butyl)amino]propionitrile

According to the method described in Example 44. reacting 1.63 g (7.02 mmol) N-(3,4-dichlorobenzyl)-2-methylpropan-2-amine and 0.92 ml (14 mmol) acrylonitrile, 1.5 g title compound is obtained in the form of an oil.

LC-MS[MH⁺]=285 (C₁₄H₁₈Cl₂N₂ 285,216).

### a.) N-(3,4-dichlorobenzyl)-N-(tert.-butyl)propan-1,3-diamine

0.92 g (3.23 mmol) 3-[1-(3,4-dichlorobenzyl)](tert-butyl)amino]propionitrile is hydrogenated according to the method described in Example 44. and thus 0.8 g title compound is obtained in the form of an oil.

LC-MS[MH⁺]=289 (C₁₄H₂₂Cl₂N₂ 289.248).

### b.) N-{3-[(3,4-Dichlorobenzyl)(tert-butyl)]amino]propyl}-3-(5-morpholin-4-yl[1,3]thiazolo[5,4-b]pyridin-2-yl)propanamide

According to the procedure described in Example 44. starting from 0.3 g (0.91 mmol) 3-(5-morpholin-4-yl[1,3]thiazolo[5,4-b]pyridin-2-yl)propionic acid hydrogen chloride salt and 0.26 g (0.91 mmol) N-(3,4-dichlorobenzyl)-N-(tert-butyl)propan-1,3-diamine, 440 mg title compound is obtained in the form of an oil.

LC-MS[MH⁺]=564 (C₂₇H₃₅Cl₂N₅O₂S 564.578).

### EXAMPLE 46

In known methods the tablet of the following composition is prepared:

| | |
|---|---|
| Active component: | 40 mg |
| Lactose: | 35 mg |
| Avicel: | 21 mg |
| Crospovidone: | 3 mg |
| Magnesium stearate: | 1 mg |

### EXAMPLE 47

### A.) Human Recombinant CCR³ Receptor (hr-CCR³) Binding Assay

The CCR³ receptor antagonist effect of the compounds of general formula (I) was examined on eotaxin binding test on hCCR3 receptor expressing recombinant K562 and RBL2H3 cells. To the tests Eotaxin labelled with radioactive iodine ¹²⁵I—(2200 Ci/mmol) was used.

In the assay 200000 cells are incubated in the presence of 0.11 nM ¹²⁵I-Eotaxin, incubation: 60 minutes at 37° C. Composition of the assay buffer: RPMI-1640 medium, pH=7.6 (GIBCO), [containing 80 mg CHAPS, 500 BSA (protease free), 100 mg Gelatine, 3 ml 25 mM HEPES in 100 ml RPMI]. The test compounds are dissolved in DMSO, the stock solution is diluted with the assay buffer. The final DMSO concentration is not more than 1%. The assays are performed in deep-well plates. The cells are incubated with the test compounds for 15 minutes, then the labelled eotaxin is added. The non-specific binding is determined in the presence of 200 nM non-labelled eotaxin. After 1 hour of incubation, 500 μl ice-cold assay buffer containing 0.5 M NaCl solution is added. The reaction is terminated by centrifugation in plate centrifuge (JUAN) at 3600 g for 6 minutes. The supernatants are poured off by turning the plates in upside-down position. The remaining droplets were blotted with tissue paper. For solubilization 200 μl 0.5 M NaOH solution is added to the pellets. After 1 hour of solubilization at room temperature the radioactivity of 150 μl solubilized solution is counted in gamma counter (1470 Wizard, Wallac).

The radioactivity of the solution is in direct ratio with the number of the receptors of the cells, with the amount of the bound ¹²⁵I-Eotaxin and with the activity of the tested antagonist.

The specific binding is calculated as the difference between the total and the non-specific bindings. The activity of the compounds is calculated from the specific binding and from the binding measured in the presence of the antagonist molecule.

The activity of the compounds is characterized with the IC₅₀ value.

### B.) Investigation of Ca²⁺ Mobilization in hCCR3-RBL and hCCR3 K562 cells

HCCR3-K562 and hCCE3-RBL2H3 cells in 40000 cells/well density (number of cells in one well of the microplate) are cultured for 24 hours. The cells are washed and loaded with calcium indicator dye (Calcium Plus assay Kit, Molecular Devices). The cells are incubated in the presence of the dye for 60 minutes while loading takes place. The dye is a fluorescent calcium indicator, which sensitively indicates the intracellular calcium concentration. The intracellular calcium concentration is in direct ratio with the fluorescent signal of the sample. The experiments are performed in a BMG NOVOSTAR apparatus, at excitation and emission wavelengths.

The selective agonists used in the experiments are:

Eotaxin

Eotaxin-2

Eotaxin-3

RANTES

Following the addition of the selective agonist, the intracellular calcium concentration in the cells significantly increases which can be monitored with the help of the fluorescent signal. In the experiments an agonist concentration is used which causes a 75% calcium signal compared to the maximum attainable signal.

Antagonists are added 15 minutes before the agonist treatment.

The change of the fluorescent signal is monitored for 30 seconds, during that period the process takes place.

The intensity of the maximum signal following the addition of the agonist is compared with the calcium signal obtained after the addition of the same agonist, but in the presence of the inhibitor.

The activity of the compounds is characterized with the IC₅₀ values.

On the basis of tests A and B the compounds of general formula (I) were found biologically active. The most potent compounds are the compounds of general formula (I) according to claim 2, which form a narrower group of the compounds of general formula (I) according to claim 1. Their IC₅₀ values are in the range of 0.5 nM to 500 nM. Of these compounds, the especially favoured molecules have IC₅₀ values between 0.5 nM and 15 nM.

1. A compound of the general formula (I), 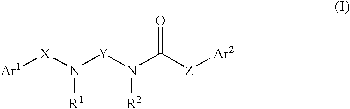
wherein
Ar¹ stands for phenyl group, optionally substituted with one or more halogen atom;
X and Y independently mean straight C₁₋₄ alkylene group, optionally substituted with one or more identical or non-identical straight or branched C₁₋₄ alkyl group;
Z means valence bond or straight C₂₋₄ alkylene group or straight C₂₋₄ alkenylene group, optionally substituted with one or more identical or non-identical straight or branched C₁₋₄ alkyl group;
R¹ and R² independently mean hydrogen atom or straight or branched C₁₋₄ alkyl group;
Ar² stands for phenyl group, thienyl group or furyl group, each optionally substituted with one or more identical or non-identical substituents selected from the group consisting of straight or branched C₁₋₄ alkyl group, straight or branched C₁₋₄ alkoxy group, hydroxyl group, amino group, amino group—substituted with one or two identical or non-identical straight or branched C₁₋₄ alkyl group-, trifluoromethyl group, cyano group, C₁₋₂ alkylenedioxy group, and halogen atom;
5- or 6-membered heterocyclic ring group containing one, two, or three nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, straight or branched C₁₋₄ alkoxy group, halogen atom, nitro group, cyano group, carboxyl group, phenyl group—optionally substituted with one or more straight or branched C₁₋₄ alkyl group, halogen atom, or benzyloxy group-, oxo group, —NR¹⁰R¹¹ group, —CONR¹⁰R¹¹ group, —SO₂NR¹⁰R¹¹ group, wherein R¹⁰ and R¹¹ independently mean hydrogen atom, straight or branched C₁₋₄ alkyl group, C₃₋₆ cycloalkyl group, or benzyl group, or R¹⁰ and R¹¹ form together with the nitrogen atom mean a group of the general formula (a), 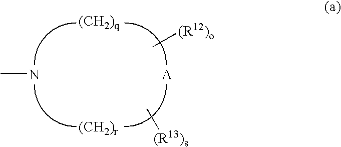
wherein
R¹² and R¹³ stand for hydrogen atom or straight or branched C₁₋₄ alkyl group,
A stands for methylene group, oxygen atom, sulphur atom, or —NR¹⁴— group—wherein R¹⁴ stand for hydrogen atom, straight or branched C₁₋₄ alkyl group, C₃₋₆ cycloalkyl group or benzyl group-,
q represents zero, 1, 2, or 3,
r represents 1, or 2,
o represents zero, or 1,
s represents zero, or 1;
benzologue of the 5- or 6-membered heterocyclic ring group wherein the benzene ring may optionally be further substituted with one or more identical or non-identical substituents selected from the group consisting of halogen atom, straight or branched C₁₋₄ alkyl group, straight or branched C₁₋₄ alkoxy group, trifluoromethyl group, nitro group, cyano group, carboxyl group, C₁₋₂ alkylenedioxy group, hydroxyl group, sulfonyl group, —NR¹⁰R¹¹ group, —CONR¹⁰R¹¹ group, and —SO₂NR¹⁰R¹¹ group; or
5- or 6-membered heterocyclic ring group containing one, two or three nitrogen atoms, or one nitrogen atom and one oxygen atom, or one nitrogen atom and one sulphur atom, condensed with 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, cyano group, carboxyl group, hydroxyl group, —NR¹⁰R¹¹ group, —CONR¹⁰R¹¹ group, and —SO₂NR¹⁰R¹¹ group; or
a salt, solvate or isomer thereof, salt of the solvate or isomer thereof, or solvate of the isomer thereof.

2. The compound of the general formula (I) according to claim 1, wherein
Z means straight C₂₋₄ alkylene group or C₂₋₄ alkenylene optionally substituted with one or more identical or non-identical straight or branched C₁₋₄ alkyl group;
Ar² stands for phenyl group;
5- or 6-membered heterocyclic ring group containing one, two, or three nitrogen atoms, or one nitrogen atom and one oxygen atom, or one nitrogen atom and one sulphur atom, optionally substituted with one or more straight or branched C₁₋₄ alkyl group;
benzologue of the 5- or 6-membered heterocyclic ring group wherein the benzene ring may optionally be further substituted with one or more identical or non-identical substituents selected from the group consisting of halogen atom, straight or branched C₁₋₄ alkyl group, amino group, amino group—substituted with one or more identical or non-identical straight or branched C₁₋₄ alkyl group-; or
5-membered heterocyclic ring group containing two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, —CONR¹⁰R¹¹ group, and —NR¹⁰R¹¹ group; or
a salt, solvate or isomer thereof, salt of the solvate or isomer thereof, or solvate of the isomer thereof.

3. The compound of the general formula (I) according to claims 1 selected from
3-(Benzothiazol-2-yl)1-N-{3-[(3,4-dichlorobenzyl)(methyl)amino]propyl}propanamide,
N-{3-[(3,4-dichlorobenzyl)(methyl)amino]propyl}-3-(6-methylbenzothiazol-2-yl)-propanamide,
N-{3-[(3,4-dichlorobenzyl)(methyl)amino]propyl}-3-(6-methylbenzoxazol-2-yl)propanamide,
3-(1H-Benzimidazol-2-yl)-N-{3-[(3,4-dichlorobenzyl)(methyl)amino]propyl}propanamide,
N-{3-[(3,4-dichlorobenzyl)(methyl)amino]propyl}-3-phenylpropanamide,
N-{3-[(3,4-dichlorobenzyl)(methyl)amino]propyl}-3-(7-methyl-[1,2,4]triazolo[1,5-a]pyridin-2-yl)propanamide,
N-{3-[(3,4-dichlorobenzyl)(methyl)amino]propyl}-3-(5-dimethylaminothiazolo[5,4-d]pyrimidin-2-yl)propanamide,
N-{3-[(3,4-dichlorobenzyl)(methyl)amino]propyl}-3-(5-dimethylaminothiazolo[5,4-b]pyridin-2-yl)propanamide,
N-{3-[(3,4-dichlorobenzyl)(methyl)amino]propyl}-3-(5-isopropylaminothiazolo[5,4-b]pyridin-2-yl)propanamide,
N-(3-{[1-(3,4-dichlorophenyl)ethyl]amino}propyl)-3-(5-methylamino[1,3]thiazolo[5,4-b]pyridin-2-yl)propanamide,
N-{3-[[1-(3,4-dichlorophenyl)ethyl])(methyl)amino]propyl}-3-(5-methylamino[1,3]thiazolo[5,4-b]pyridin-2-yl)propanamide,
N-{3-[[1-(3,4-dichlorophenyl)ethyl])(methyl)amino]propyl}-3-(5-piperidin-1-yl[1,3]thiazolo[5,4-b]pyridin-2-yl)propanamide,
N-{3-[[1-(3,4-dichlorophenyl)ethyl])(methyl)amino]propyl}-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-piperidin-1-yl[1,3]thiazolo[5,4-b]pyridin-2-yl)propanamide,
N-(3-{[1-(3,4-dichlorophenyl)ethyl]amino}propyl)-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-morpholin-4-yl[1,3]thiazolo[5,4-b]pyridin-2-yl)propanamide
N-{3-[(3,4-dichlorobenzyl)(isopropyl)]amino]propyl}-3-(5-morpholin-4-yl[1,3]thiazolo[5,4-b]pyridin-2-yl)propanamide, or
N-{3-[(3,4-dichlorobenzyl)(tert-butyl)]amino]propyl}-3-(5-morpholin-4-yl[1,3]thiazolo[5,4-b]pyridin-2-yl)propanamide; or
a salt, solvate or isomer thereof, salt of the solvate or isomer thereof, or solvate of the isomer thereof.

4. A process for the preparation of the compounds of the general formula (I) according to claim 1 comprising,
1) reacting a diamino compound of the general formula (III), wherein Ar¹, X, Y, R¹, and R² are as defined in claim 1 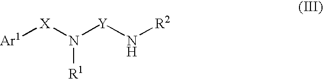
with a carboxylic acid derivative of the general formula (II), wherein Ar², and Z are as defined in claim 1, and W stands for halogen atom, hydroxyl group, —O(C₁₋₄alkyl) group or —OCO-Z-Ar² 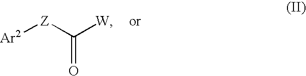
2) reacting an amino compound of the general formula (VI), wherein Ar¹, X, and R¹ are as defined in claim 1 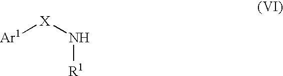
with a halogen compound of the general formula (XVII), wherein Y, R²Z, and Ar² are as defined in claim 1, and Hal means halogen atom 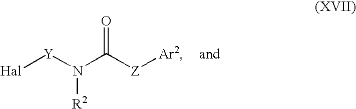
optionally transforming a substituent of the compound of the general formula (I) thus obtained according to step a), b) or c) into another by using a known method and/or the resultant compound of the general formula (I) obtained according to step a, or b is transformed into a salt or solvate thereof, or liberated from a salt or solvate thereof and/or resolved into an optically active isomer, or the optically active isomer is transformed into the racemic compound and if desired separating structural isomers from each other.

5. The process according to claim 4 a) wherein W is halogen atom which is chloride atom.

6. The process according to claim 5 wherein the reacting is carried out in the presence of an organic base.

7. The process according to claim 4 a) wherein W is hydroxyl group, and the reacting is carried out in the presence of an activating agent.

8. The process according to claim 7 wherein the activating agent is dicyclohexyl carbodiimide, pivalyl chloride, ethyl chloroformate, isobutyl chloroformate, carbonyl diimidazole, or benzotriazol-1-yl-oxy-tripyrrolidinophosphonium hexafluoro phosphate.

9. A pharmaceutical preparation wherein it contains one or more of the compounds of the general formula (I) according to claim 1, or a salt, solvate or isomer thereof, salt of the solvate or isomer thereof, or solvate of the isomer thereof and one or more excipients used in the pharmaceutical industry.

10. The pharmaceutical preparation according to claim 9, wherein the one or more compounds of the general formula (I) is/are selected from
3-(Benzothiazol-2-yl)1-N-{3-[(3,4-dichlorobenzyl)(methyl)amino]propyl}propanamide,
N-{3-[(3,4-dichlorobenzyl)(methyl)amino]propyl}-3-(6-methylbenzothiazol-2-yl)-propanamide,
N-{3-[(3,4-dichlorobenzyl)(methyl)amino]propyl}-3-(6-methylbenzoxazol-2-yl)propanamide,
3-(1H-Benzimidazol-2-yl)-N-{3-[(3,4-dichlorobenzyl)(methyl)amino]propyl}propanamide,
N-{3-[(3,4-dichlorobenzyl)(methyl)amino]propyl}-3-phenylpropanamide,
N-{3-[(3,4-dichlorobenzyl)(methyl)amino]propyl}-3-(7-methyl-[1,2,4]triazolo[1,5-a]pyridin-2-yl)propanamide,
N-{3-[(3,4-dichlorobenzyl)(methyl)amino]propyl}-3-(5-dimethylaminothiazolo[5,4-d]pyrimidin-2-yl)propanamide,
N-{3-[(3,4-dichlorobenzyl)(methyl)amino]propyl}-3-(5-dimethylaminothiazolo[5,4-b]pyridin-2-yl)propanamide,
N-{3-[(3,4-dichlorobenzyl)(methyl)amino]propyl}-3-(5-isopropylaminothiazolo[5,4-b]pyridin-2-yl)propanamide,
N-(3-{[1-(3,4-dichlorophenyl)ethyl]amino}propyl)-3-(5-methylamino[1,3]thiazolo[5,4-b]pyridin-2-yl)propanamide,
N-{3-[[1-(3,4-dichlorophenyl)ethyl])(methyl)amino]propyl}-3-(5-methylamino[1,3]thiazolo[5,4-b]pyridin-2-yl)propanamide,
N-{3-[[1-(3,4-dichlorophenyl)ethyl])(methyl)amino]propyl}-3-(5-piperidin-1-yl[1,3]thiazolo[5,4-b]pyridin-2-yl)propanamide,
N-{3-[[1-(3,4-dichlorophenyl)ethyl])(methyl)amino]propyl}-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-piperidin-1-yl[1,3]thiazolo[5,4-b]pyridin-2-yl)propanamide,
N-(3-{[1-(3,4-dichlorophenyl)ethyl]amino}propyl)-3-(5-pyrrolidin-1-yl[1,3]thiazolo[5,4-b]pyridin-2-yl)propanamide,
N-(3-{[1-(3,4-dichlorophenyl)ethyl]amino}propyl)-3-(5-morpholin-4-yl[1,3]thiazolo[5,4-b]pyridin-2-yl)propanamide
N-{3-[(3,4-dichlorobenzyl)(isopropyl)]amino]propyl}-3-(5-morpholin-4-yl[1,3]thiazolo[5,4-b]pyridin-2-yl)propanamide, and
N-{3-[(3,4-dichlorobenzyl)(tert-butyl)]amino]propyl}-3-(5-morpholin-4-yl[1,3]thiazolo[5,4-b]pyridin-2-yl)propanamide.

11. A method of treatment of a pathology in a patient wherein a CCR3 receptor plays a role in the development of the pathology comprising administering to the patient a pharmaceutically effective amount compound of the general formula (I) according to claim 1.

12. The method according to claim 11 wherein the pathology is selected from asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.

13. A compound of the general formula (IIa), 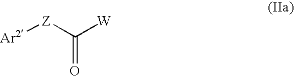
wherein
Ar^{2′} represents a 1,2,4-triazolo[1,5-a]pyridine- or thiazolo[5,4-b]pyridine group optionally substituted with one or more straight or branched C₁₋₄ alkyl group, straight or branched C₁₋₄ alkoxy group, hydroxyl group, —NR¹⁰R¹¹ group, —CONR¹⁰R¹¹ group, or —SO₂NR¹⁰R¹¹ group, wherein the meanings of R¹⁰ and R¹¹ are as defined in claim 1;
Z represents 1,3-propylene group; and
W stands for halogen atom, hydroxyl group, —O(C₁₋₄allyl) group or —OCO-Z-Ar², wherein the meanings of Z and Ar² are as defined in claim 1.
